(19) United States
(12) Patent Application Publication
(10) Pub. No.: US 2009/0252760 A1
(43) Pub. Date: Oct. 8, 2009

(54) Construction of Recombinant Adenovirus With Genes That Codify for SAG1, SAG2 and SAG3

(76) Inventors: Ricardo Tostes Gazzinelli, Minas Gerais (BR); Oscar Bruna-Romero, Minas Gerais (BR); Bráulia Costa Caetano, Minas Gerais (BR)

Correspondence Address: David H. Voorhees;Merek, Blackmon & Voorhees, 673 S. Washington St., Alexandria, VA, 22314, US

(21) Appl. No.: 12/084,434

(22) PCT Filed: Nov. 1, 2006
(86) PCT No.: PCT/BR2006/000241

§ 371 Date: May 1, 2008

(30) Foreign Application Priority Data
Nov. 1, 2005 (BR) PI 0504782-0

(51) Int. Cl. A61K 39/12; C12N 15/74; A61P 37/04

(52) U.S. Cl. 424/199.1; 435/471

(57) ABSTRACT

This invention refers to the construction of recombinant adenovirus with genes that codify for T. gondii SAG1, SAG2 and SAG3, through a homologue recombination technique between two vectors. A first vector that serves as transference vector of genes and a second vector that bears the adenoviral receptor genome of these genes. The invention is also related to the use of recombinant adenovirus in a vaccine composition to obtain the immunization against infections caused by T. gondii parasite.

### FIELD OF THE INVENTION

This invention refers to a recombinant adenovirus and to a construction process of a recombinant adenovirus with genes that codify for SAG1, SAG2 and SAG3 proteins of T. gondii parasite, using the homologue recombination technique between two transfer vectors. Additionally, this invention describes a vaccine composition using a recombinant adenovirus and an immunization method against infections caused by T. gondii parasite.

### RELATED TECHNIQUES

Toxoplasmosis is a disease caused by the Toxoplasma gondii protozoa. The referred protozoon is a necessary intracellular parasite pertaining to the Apicomplexa phylum. The referred protozoon is able to infect any type of nucleated cell of the body, specially central nervous system cells, endothelia and striated muscles, as for instance muscle-skeletal cells and heart cells (myocardium).

The T. gondii protozoon presents a facultative heteroxen life cycle and has felines as definite hosts, however, the said protozoon is very promiscuous regarding its intermediate host because of intermediate host can be man or other hot-blooded animals (homoeothermic). The T. gondii protozoa cycle starts with the invasion of the small intestine cells of felines, where the intra-epithelial cycle occurs, characterized by the sexual reproduction of the of the parasite, called gametogonic, or also known as the gametogonic cycle. In this cycle differentiated masculine and feminine gametes appear, giving origin to oocysts that are releases in the felines' faeces.

The oocysts, in order to become infective to other animals, suffer the sporulation process, therefore one of the oocysts gives origin to two sporocysts, each one of which, on their turn, give origin to four sporozoites. These infecting oocysts are able to survive the gastric digesting, to sulfuric acid at 1% concentration, to potassium dichromate at 2.5% and to hypochlorite at 2.5% concentration, and they are able to maintain themselves viable for many months in a moisty and airy environment.

After ingestion of water or of food contaminated by the infecting oocysts by the intermediate host or by the feline itself, the parasite is released into the cell as sporozoites and they penetrate the intestinal mucosa, where they rapidly reproduce themselves. Once at the intestinal mucosa, sporozoites suffer cell divisions, generating tachyzoites, which invade other cells, including body defense cells, as for instance macrophages.

Macrophages, as mobile cells and since they circulate with the blood, carry the parasite to the regional lymphatic glanglions, where the multiplication process continues. Thus, the parasite can be disseminated throughout the body.

Tachyzoites are the asexual form of the parasite, which proliferates quickly, thus disseminating through blood circulation or other tissues of the body, where they invade nucleated cells. Tachyzoites present several structures common to other animal cells, as for instance, mitochondria, the endoplasmatic reticulum and the Golgi complex, besides presenting small organs characteristic of the phylum, as for instance polar rings, conoid, rhoptries and micronema. Some tachyzoites, after perfusion of the host cell develop more slowly, forming bradyzoites, which shall form tissue cysts.

Intact cysts do not cause any damage to the intermediate host and can remain for long periods or for their entire life in the intermediate host without causing any inflammatory response or arising a significant tissue response on it.

The destination of tissue cysts is not completely known, but it is supposed that, sometimes, cysts disrupt themselves during their life cycle in order to release bradyzoites, which can be destroyed by the immune system of the intermediate host or form new cysts.

The wall of the cyst is dissolved by human digestive enzymes and the bradyzoites released are able to perfuse intestine epithelial cells, thus disseminating throughout the individual in case of intermediate hosts and in definite host there is the formation of oocysts, which are faeces excreted, therefore they can contaminate the environment and other animals.

Humans acquire T. gondii infection mainly through the ingestion of oocysts present in water and food accidentally contaminated by felines or through ingestion of meat or raw or poorly cooked entrails from chronically infected animals and bearers of tissue cysts.

Other less frequent forms of contamination by T. gondii parasite occur by organ grafts, laboratory accidents and blood transfusion from individuals during the acute infection stage.

Toxoplasmosis is one of the most common zoonosis in several world regions with a medical and veterinary significance. It is deem that one third of the world population is infected by T. gondii. Despite the elevated frequency of infection within populations, the occurrence of clinical toxoplasmosis signs is low, since the (post natal) infection acquired in immune competent individuals leads to an effectively immune response to the control of the parasite. However, there are reports of ocular damage in known infected individuals during their adult life.

Toxoplasmosis acquired bears a higher impact on individuals with the immune system commitment. Toxoplasmosis in incompetent patients is generally benign, however, due to immune system impairment, a chronic infection can be reactivated and unfold in a serious form.

In serum positive patients, the protozoon is responsible for the development of a variety of symptoms of the disease; however, the most frequent symptom is encephalitis, in which the quick multiplication of tachyzoites results in neural tissues destruction. An important feature in these patients is the possible reactivation of the infection due to the present of latent cysts. It is estimated that 40% of patients with HIV virus develop encephalitis by toxoplasma, and 10 to 30% of these patients die.

Disseminated toxoplasmosis can also be a complicating factor in organ and bone marrow grafts, resulting both from the organ graft from T. gondii infected donors for a susceptible donor, as from the reactivation of the latent infection in receptors due to immunosuppressant therapy.

However, the major complications related to toxoplasmosis derive from congenital or pre natal infection. The transplacental transmission of the T. gondii protozoa occurs when the pregnant individual makes a first contact with the referred protozoon during pregnancy.

If the primary infection occurs before pregnancy, the acquired immunity impedes the transmission of the protozoa to the fetus in subsequent exposures. However, if the pregnant woman suffers an impairment of the immune function, the fetus can be infected. Within the last three decades, the pre natal infection incidence has been estimated between 1 and 100 for each 10,000 births in different countries. The transmission risk increases with gestation time, from 14% during the first quarter to 59% during the last.

On the other hand, infection effects on the fetus are reduced during pregnancy, thus, most neonates are asymptomatic at birth, but frequently present sequels due to congenital infection throughout life.

Toxoplasmosis has also been frequently diagnosed in patients affected by cancer, which are under intense treatment with chemotherapeutics. Among diagnosed complications in cancer patients, there is the onset of chorioretinitis associated to encephalitis, as well as complications in other organs.

A second relevant aspect of toxoplasmosis refers to economic losses in livestock. Mainly in porcine and ovine culture, the T. gondii infection causes great losses due to abortion, stillborns and fetal malformations. Therefore, the congenital transmission of toxoplasmosis also bears a significant impact in the production index of porcine and ovine cattle.

At the installation of the parasite in the body, the immune response process by individual's immune system begins. This process comprises a primary immune response, which consists in an innate non-specified immunity, mediated by macrophages and natural killer (NK) cells. Additionally, the referred primary response is characterized by the onset of the first circulating antibodies, the M type immune globulins (IgM) with elevated molecular weight (19 S), and which are able to mediate agglutination, lytic or complement fixation mechanisms. Then, the system produces a retarded immunity, which is mediated by the endothelial reticulum system cells, characterized by the onset of IgG antibodies, with low molecular weight (7 S).

NK cells are activated directly by products of the parasite and secrete interferon gamma (IFN-γ). Macrophages are also directly stimulated by products of the parasite and secrete interleukin 12 (IL-12). IL-12 together with a tumoral necrosis factor (TNF) act jointly with IL-1β and IL-15 interleukins on the NK cells and additionally stimulate the IFN-γ production.

IFN-γ acts on the macrophage themselves, increasing their microbicide and IL-12 synthesis capacity. Another property of IFN-γ is to activate the synthesis of chemokines involved in the recruitment of T lymphocytes and acting as IL-12 co-factor in the differentiation of Thp cells in Th1 effector cells.

The regulation of the intensity of the immunological response and of the inflammation caused by the T. gondii parasite is performed by the macrophages of the immune system themselves, which also produce anti-inflammatory typo 2 cytokines, as for instance IL-10 that inhibits the IL-12 synthesis by the macrophages and the IFN-γ production by NK cells and Th1 lymphocytes, and the IL-4, which potentializes the effect of IL-10 on macrophages and induces the differentiation of Thp precursors in effector cells with a Th2 profile.

As the immune response develops by the immune system of the host, the elimination of the tachyzoites forms occur, with the onset of the bradyzoites forms and the formation of tissue cysts.

However, the cystic forms of the parasite shall persist for many years and even throughout the life of the infected host, if the same shall not perish from the illness. In these situations, the individual becomes an asymptomatic bearer of the parasite, with an elevated title of antibodies in its body. If the cysts shall multiply in the interior of the cells, in a slow way, the antigenic stimulus is maintained and only if intercurrent diseases shall occur or the use of immune-suppressive medication can cause the reactivation of toxoplasmosis, with the breakage of such cysts and their leak to the neighboring pericystic tissue.

During the chronic stage of the infection, cytokines levels are reduced and the maintenance of the infection control becomes dependent from the development of the acquired immunity by means of the activation of specific T cells.

Peptides derived from the T. gondii parasite are presented to lymphocytes, associated to molecule of the Main Histocompatibility Complex (MHC) and co-stimulating molecules, in an environment rich in type 1 cytokines.

The result of this presentation of peptides is the differentiation of T CD4⁺ and T CD8⁺ lymphocytes in cells producers of IFN-γ, in such a way that effector lymphocytes T CD8⁺ also develop a cystolic activity on the cells infected by the T. gondii parasite.

In a similar way to what happens in the acute stage of the infection, IFN-γ represents a significant role in the chronic stage of the infection. In the chronic stage in macrophages, this (IFN-γ) cytokine activates the induced nitric oxide syntax (iNOS) and the synthesis of moderate levels of reactive nitrogen intermediates (RNI), thus resulting in an increase in the microbicide capacity of macrophages. IFN-γ yet assists on the differentiation of the cytotoxic T lymphocytes; increases the MHC expression in infected cells and favors the differentiation of B-lymphocytes in cells that produce IgG1 (humans) or IgG2a (rats). Although Antibodies do not have a significant role in the initial response to the pathogen, they can exercise different functions during the chronic phase, such as the opsonization, fixation of the complement, or mediation of the antibodies dependent cytotoxic effect.

Presently, the prevention and control of toxoplasmosis depend on epidemiological surveillance measures, sanitary handling and inspection. These actions aim at identifying and eliminating infection sources, in order to reduce the transmission index of T. gondii within groups of risk, such as women during fertile age and immune-affected patients.

The measures for the prevention of the infection caused by T. gondii are not sufficient to avoid the occurrence of toxoplasmosis onsets, and despite the disease be treatable with antibiotics, the quick development of resistance to chemical bases by the parasite restricts the amount of medications available for treatment.

Thus, the use of anti-toxoplasma vaccines is a significant alternative for the control of the disease. The main objective of a vaccine against T. gondii is to generate an immunological response within the several hosts, therefore, controlling the replication of the parasite and its transmission within a population.

Studies of an immune response through infection with attenuated T. gondii strains lead to the development of the first commercial vaccine against the parasite, for use in ovines. This vaccine is a compost of the modified tachyzoite forms of the S48 strain, which, after subcutaneous inoculation in non-immune sheep makes that the tachyzoites multiply into local lymphonodes thus causing a benign fever response. The peak of the antibodies title is reach within six weeks. The immunity conferred by this vaccine probably involves a cell response with activation of T CD4⁺ and T CD8⁺ lymphocytes, with a Th1 profile.

Other strains attenuated through radiation—such as ts-4, a temperature sensible mutant—demonstrated to be efficient in the creation of a cell immune response and protection against the challenge with T. gondii pathogenic strains in rats. However, the use of such immunogens in humans is improbable due to the fact that the tachyzoites present in their composition are alive and they can revert to a pathogenic status and cause the disease.

An illustration of the method for the production of antibodies for T. gondii caused infection is described in document EP 638.316 (corresponding to PI9403202). This document describes a method for the production of antibodies or immunity mediated by cell to an infectious organism in a hot blooded mammal that comprises the administration through the nose, intramuscular or subcutaneous to this hot blooded mammal, of live recombinant adenovirus, where the vironic structural protein is unaltered in relation to the native adenovirus from each the recombinant adenovirus is produced, and that contains the gene that codifies to the correspondent antigen, the referred antibodies or in a way that induces the said cell mediated immunity.

A vaccine composition is also described in the document WO 01/43768, where there is a description of a vaccine compost comprising of a toxoplasma protein, SAG3 with an (S) sequence of 385 amino acids, or their immunogenic derivate, combined to an adequate adjuvant and/or supportive. The referred document describes a toxoplasma-truncated protein—SAG3, preferably purified, where an anchor region of the SAG3 is absent. The term “immunogenic derivate” comprises any molecule, as for instance a truncated or another protein derivate, which retains the ability to induce a protein immune response due to the internal administration in humans, or in an animal, or which retains the ability of reacting with antibodies present in the serum or in another biological sample of Toxoplasma gondii infecting humans and animals. These immunogenic derivates can be prepared by the addition, deletion, substitution or rearrangement of the amino acid or through chemical modifications of it.

An effective vaccine enables the development of immunity before the infection caused by the parasite, thus avoiding the release of oocysts by definite hosts. Besides, the referred effective vaccine can prevent the formation of tissue cysts in intermediate hosts and their oral transmission through the ingestion of contaminated food. A vaccine could also prevent acute toxoplasmosis in pregnant women and the transplacentary transmission.

The construction of a recombinant adenovirus requires differentiated techniques of molecular biology, in a such way that two recombinant adenovirus with ability to express the same protein can be completely different, as well as their ability to protect against the corresponding infection, due to the pattern of their molecular construction.

Another peculiarity resides in the fact that what makes a specific recombinant adenovirus function as a therapeutic tool is not only the fact that the virus is generated, but a set of factors that include the correct design of the artificial viral genome constructed, the type of recombinant virus generated and the inclusion of DNA sequences that are able to codify the antigen in the correct position at the viral genome.

### SUMMARY OF THE INVENTION

In general, the invention presents the construction of recombinant adenovirus with genes that codify for T. gondii SAG1, SAG2 and SAG3 proteins, through the technique or homologue recombination between two transference vectors.

A first objective of this invention is to provide recombinant adenoviruses with the genes that codify for T. gondii SAG1, SAG2 and SAG3 proteins

Another objective of the invention refers to a construction process of recombinant adenovirus with genes that codify for T. gondii SAG1, SAG2 and SAG3 proteins, through the homologue recombination technique between two transference vectors.

In addition, another objective of the invention is to present vaccine compost using the recombinant adenovirus.

Another invention objective is to provide an immunization method against infections caused by parasite T. gondii.

The mechanism of action of vaccines based on recombinant adenoviruses consists in the infection of host cells and intracytoplasmatic expression of heterologue antigens by infected cells, which can or cannot be followed by secretion of the produced antigen. The recombinant adenoviruses are able to infect immune cells specialized in the presentation of antigens, such as dendritic cells, which render these vectors highly immunogenic.

Besides that, some proteins that form the viral capsid have an immune stimulating effect, that is, they are able to potentialize the immune response, as well as to help in the direction of the profile of cytokines produced by the effector cells for a Th1 type, which is important in the protection against parasitic agents as the Toxoplasma gondii.

### BRIEF DESCRIPTION OF DRAWINGS

FIG. 1 shows the general plan of the construction process of recombinant virus.

FIG. 2A shows reaction initiators designed for amplification of genes without the 3′ portion.

FIG. 2B shows initiators of reaction of amplification of SAG3 protein.

FIG. 3 shows the presence of the recombinant gene and the expression of SAG1 and SAG2 proteins.

FIG. 4 shows stages of SAG3 cloning, with the substitution of the original signal peptide of the gene for a signal viral peptide.

FIG. 5 shows the expression of the different SAGs proteins with recombinant adenovirus.

FIG. 6A shows a comparison of the results of the ELISA testes of serum of immunized animals with AdSAG1 face antigens of the T. gondii protozoa, related to the presence of the IgG antibody.

FIG. 6B shows a comparison of the results of ELISA tests of serum of animals immunized with AdSAG2 face antigens of the T. gondii protozoa, related to the presence of the IgG antibody.

FIG. 6C shows a comparison of the results of ELISA tests of serum of animals immunized with AdSAG3 face antigens of the T. gondii protozoa, related to the presence of the IgG antibody.

FIG. 6D shows a comparison of the results of ELISA tests of serum of animals immunized with a combination of AdSAG1, AdSAG2, AdSAG3 face antigens of the T. gondii protozoa, related to the presence of the IgG antibody.

FIG. 7A shows the results of the count of the number of cysts present within the brain of BABLB/c rats in the experiment with AdSAG1, in the experiment with AdSAG2, and in the experiment with a combination of the two vectors.

FIG. 7B shows the results of the count of the number of cysts present within the brain of BABLB/c rats in the AdSAG3 experiment and in the experiment with a combination of the three vectors.

### DETAILED DESCRIPTION OF THE INVENTION

This substantiation describes a construction process of recombinant adenovirus with genes that codify for T. gondii SAG1, SAG2 and SAG3 proteins. This construction process occurs by means of the homologue recombination technique between two vectors (plasmids). This homologue recombination technique uses:
a transference vector, which can be any plasmid that contains an expression cassette adapted to eukaryote cells sided by homologue sequences of preferably type 5 human adenovirus. Additionally, plasmids that present sequence of other human adenovirus, such as 1, 2, 4 and 7 adenovirus, and plasmids that present sequences of monkey adenovirus such as SA7P, 1, 22, 23, 24 and 25 could also be employed in the homologue recombination technique, because the adenoviruses exemplified present a high similarity degree of similarity with type 5 human adenovirus, which was employed in this substantiation; and
a vector containing a complete genome of the human adenovirus, preferably type 5, as for instance, pJM17.

In this substantiation, pCMVlink1 and pMV60 plasmids were alternatively employed as transference vectors. Each one of these plasmids presents an expression cassette, where there are multiple cloning sites. In pCMVlink1 plasmid, this cassette is composed by a cytomegalovirus promoter and a polyadenilation sequence of the SV40 virus. In pMV60 plasmid, the expression cassette is composed by a cytomegalovirus promoter and a polyadenilation sequence of cytomegalovirus.

The transference vectors (plasmids) also present sequences of adenovirus, preferably of type 5, sided by the expression cassette and by a gene resistant to ampicillin.

The pJM17 plasmid consists of a genome of the modified human adenovirus type 5. This modification occurs due to the insertion of a segment of exogenous DNA, as for instance pBRX plasmid, in a region of the viral genome viral, specifically, region E1. This region E1 contains the genes that codify the proteins responsible for the replication of the viral genome and the insertion of the exogenous DNA breaks these Region E1 genes. Therefore, the insertion of the pBRX plasmid in region E1 renders pJM17 deficient in replicating.

The pJM17 plasmid can only be replicated specifically in HEK293 species cells. Such cells were transformed with fragments of DNA from human type 5 adenovirus derived from region E1 of the viral genome. Thus, HEK293 cells express proteins responsible for the control of the viral replication and they can provide these proteins in a complementary form, allowing the replication of the modified viral genome.

Additionally, the insertion of the pBRX plasmid to region E1 makes that the size of the adenovirus genome of the adenovirus exceeds the packaging capacity of the genetic material of the viral capsid. Therefore, even if a modified pJM17 viral genome is introduced into a permissive cell, as for instance the HEK293A cell, and even if viral the genome is replicated and expressed, there shall be no assembly of new viral particles in face of the excessive size of the genome.

In the process of homologue recombination between the transference vectors and the plasmid pJM17, the expression cassette of the transference vector, which contains the gene of protein SAG, is transferred to plasmid pJM17, therefore substitution all the E1 region. This region E1 substitution results in a viral genome, which can be replicated in a permissive cell and can be packaged in the capsid for the assembly of new viral particles, since the elimination of E1 region reduces the size of the genome.

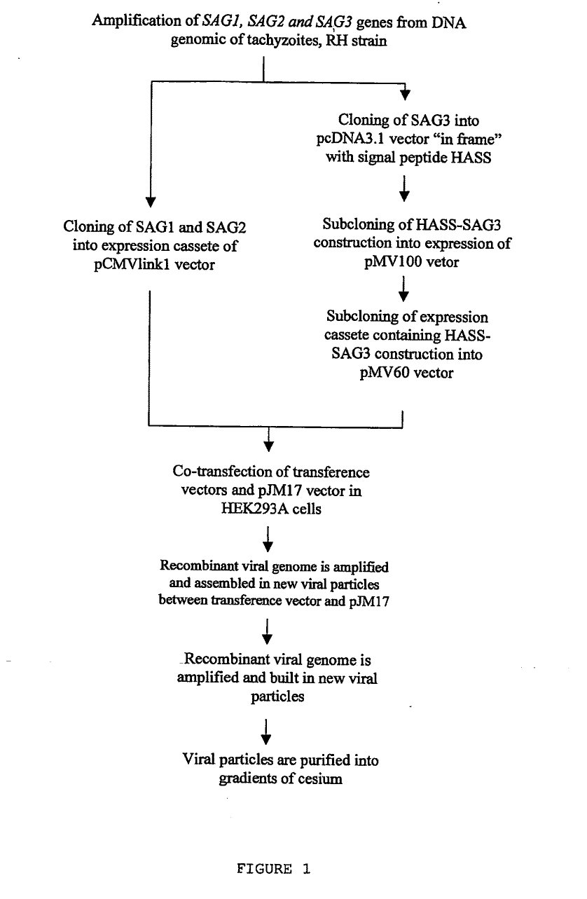

FIG. 1 shows the general plan for the construction process of recombinant virus. This construction process of recombinant virus comprises stages of:
cloning of adenovirus genes that codifies SAG1, SAG2 and SAG3 proteins in the transference vector,
co-transfection of transference vectors and of the genome of the adenovirus,
intracellular homologue recombination between the transference vectors and the genome of the adenovirus,
amplification of the recombinant viral genome viral
purification of viral particles into an adequate gradient, as for instance, a cesium gradient, and
characterization of the expression of the proteins of interest.

During the cloning stage, genes of SAG1, SAG2 and SAG3 proteins were amplified by means of chain reaction of polymerase (PCR) and as of the genomic DNA of a T. gondii strain and in the form of tachyzoites. The T. gondii strain used in this substantiation was the RH strain.

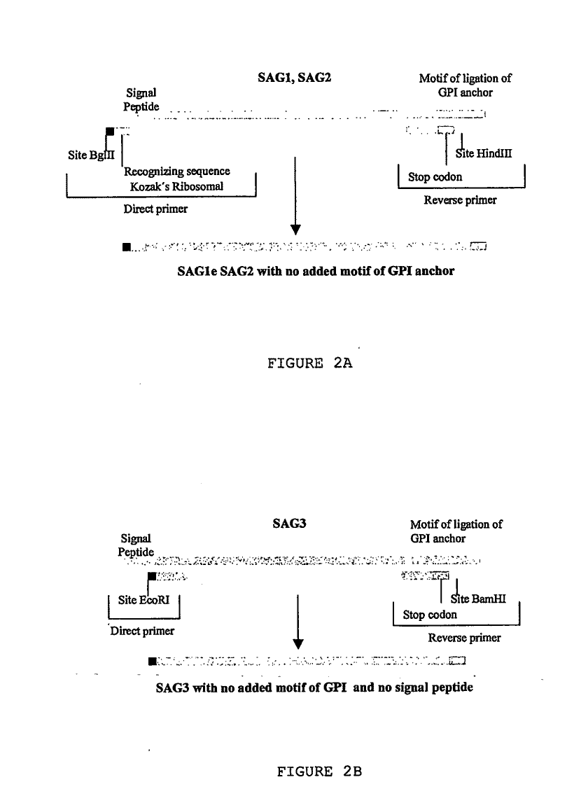

In the case of proteins SAG1 and SAG2, initiators of reaction, roughly represented in FIG. 2A, they were designed for a parameter in an internal position in SAG1 and SAG2 sequences and amplification genes of SAG 1 and of SAG2, without the 3′ portion. This 3′ portion of the SAG1 gene corresponds to the last 69 nucleotides of sequence of the referred gene and the referred 3′ portion of the SAG2 gene corresponds to the 51 last nucleotides of the referred gene. These nucleotides codify, within the respective proteins, the reason for the addition of the glycosylphosphatidylinositol (GPI) anchor.

A reason for the addition of the GPI anchor consists on 23 and 17 last amino acids of the carboxy-terminal portion of the SAG1 and SAG2 proteins, respectively, which serve as a delimitation signal for the addition of a GPI molecule. This GPI molecule is a lipidic structure linked to the SAG1 and SAG2 proteins right after the synthesis of these proteins.

The GPI molecule function is to mediate the connection of SAG1 and SAG2 proteins within the plasmatic membrane of the T. gondii parasite. The addition of the GPI anchor into SAG1 and SAG2 proteins is a mechanism peculiar of the T. gondii, which can be faithfully reproduced in a mammal host cell (where we intend to express the referred proteins). Thus, the presence of the referred reason for the addition of GPI in genes SAG1 and SAG2 could activate erroneous processing mechanisms of proteins SAG1 and SAG2 immediately after their synthesis within the mammal cells. In order to avoid the occurrence of possible processing errors that could prejudice the expression of SAG1 and SAG2 in the mammal's host cells, we decided, in this substantiation, to clone the respective genes without the reason for addition of GPI.

Restriction sites for endonuclease BglII and Kozac Nos ribossomal recognition sequence were inserted in direct initiators of PCR reaction for amplification of SAG 1 and SAG2 genes. In reverse initiators, restriction sites for HindIII and TGA stop codons were inserted.

Initiators represented in FIG. 2B were employed for amplification of the SAG3 gene. These initiators were designed to allow a pairing in internal positions of the SAG3 gene and amplification of said gene without 5′ extremity and without 3′ portion. This 3′ portions corresponds to the last 69 nucleotides of the SAG3 gene and has its characteristics and functions similar to the SAG1 and SAG2 genes. The removal of this portion obeys the same criteria used for the removal of the portion of SAG1 and SAG2 genes.

The referred 5′ portion corresponds to the first 105 nucleotides of the SAG3 gene, which codify, in protein SAG3, the signal peptide for addressing the endoplasmatic reticulum (PS). This peptide is responsible for the transport of SAG3 protein to the rough endoplasmatic reticulum immediately after the SAG3 protein synthesis.

The recognition process of the signal peptide and intracellular addressing of the SAG3 protein is a mechanism peculiar to T. gondii, which may not be faithfully reproduced by the cells of a mammal host (where we wish to express the protein). This can lead to errors in the processing of the SAG3 protein immediately after its synthesis in the cells of the mammal. In order to avoid possible errors and prejudice of the expression of SAG3 proteins, we decided to clone the SAG3 gene without the mentioned signal peptide.

The direct initiators of the amplification response of the SAG3 protein have a restriction site for endonuclease EcoRI and reverse initiators of response have a BamHI site and a TGA stop codon.

Products of the PCR response were submitted to the electrophoresis technique with agarose gel at approximately 1%, and the gel bands corresponding to each gene were eluted in gel fragments with the use of a commercial purifying kit.

After the elution of the products of PCR response, these products and the transference vectors were submitted to the digestion process through a response with its endonucleases corresponding, at a preferred rate of 10 units of enzyme/μg of DNA, at an approximate temperature of 37° C., during approximately 3 hours.

After the digestion procedures, responses of connection among the response products of the digestion were performed with an adequate enzyme, as for instance, the T4-Ligase, at an approximate temperature of 16° C. during approximately 3 hours, in such a way to maintain the molar 3:1 ratio of insert/plasmid, and preferably 10 enzyme units per response.

The connecting response products were employed in the transformation of the Escherichia coli bacterium. In this substantiation we used a chemical-competent XLI-Blue species of the Escherichia coli bacterium.

Transforming bacteria were grown in an adequate culture, which contained an adequate culture medium, such as, for instance, the LB environment. To the LB environment we added approximately 100 μg/ml of ampicillin. The referred cultivation occurred preferably in the period within 16-18 hours and recombinant plasmids were purified with commercial plasmid insulation kits. Plasmids can be alternatively purified through alkaline lysis of the recombinant bacteria with a tampon containing sodium hydroxide and dodecyl sodium sulphate, followed by the neutralization of the bacterial lysis with a tampon composed by glacial acetic acid and potassium acetate and posterior filtration of the lysis and precipitation of the plasmids with isopropanol.

SAG1 and SAG2 genes were cloned in the way oriented in the pCMVlink1 plasmid. The presence of the recombinant gene was confirmed by the observation of the digestion profile of the purified plasmid, and the expression of SAG1 and SAG2 proteins was determined through the transfection technique of eukaryote cells, as for instance HEK293, with recombinant plasmids containing SAG1 and SAG2 genes, followed by detection of proteins in the lysis of the cells transfected through “Western-blot” technique, as shown in FIG. 3.

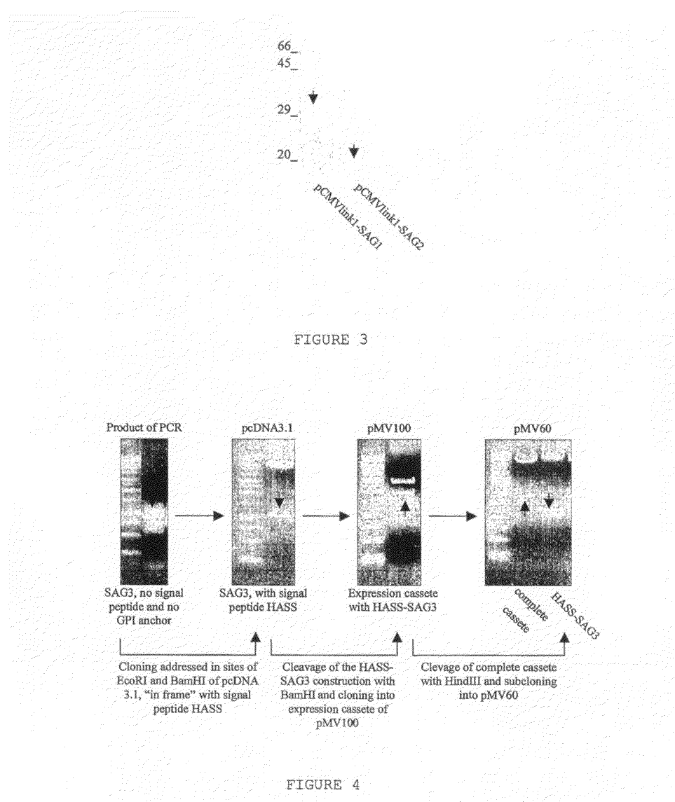

The cloning process of SAG3 gene was differentiated due to the performance of new stages during the referred process. New stages performed allowed the substitution of the original signal peptide of the gene for a viral signal peptide, as shown in FIG. 4.

With the use of scheduled initiators in FIG. 2B, the SAG3 gene was amplified, in such a way to exclude the region corresponding to the signal peptide.

Then, the SAG3 gene was inserted in the pcDNA3.1 plasmid. Before performing the cloning of the SAG3 gene, the referred pcDNA3.1 plasmid went through a preparation process, consisting of the introduction of the DNA fragment that codifies the signal peptide of haemagglutin of the influenza virus (HASS) within the expression cassette of the mentioned plasmid.

After this preparation, the SAG3 gene was also inserted in the expression cassette of the pcDNA3.1 plasmid, in a guided way. In sequence, the mentioned SAG3 gene was connected to the DNA HASS fragment, following the reading mark of the referred fragment.

The hybrid sequence HASS-SAG3 was removed from pcDNA3.1 plasmid, through cleavage with a pcDNA3.1 endonuclease. Then, the hybrid HASS-SAG3 molecule was cloned in the expression cassette of the pMV100 plasmid.

The complete expression cassette, which contains a HASS-SAG3 molecule, was removed from the pMV100 plasmid through a digestion response with an adequate enzyme, as for instance, the HindIII enzyme.

After the digestion response, the complete expression cassette containing the hybrid HASS-SAG3 molecule was cloned in the pMV60 plasmid, at a site located between two regions of homology in adenovirus present in pMV60 plasmid.

During all the cloning stages, the presence of the gene of protein SAG3 was evaluated through the profile obtained during the digestion response of plasmids and the expression of protein SAG3 by the final construction of pMV60-HASS-SAG3 molecule was evaluated through transfection of permissive cells, as for instance, HEK293A cells and detection of protein in the cellular lysis in “Western-blot”, as seen in FIG. 4.

For the homologue recombination and a construction of adenovirus, the procedure used in this invention was the co-transfection method or simultaneous transfection of 2 plasmids, through precipitation calcium chloride (CaCl₂), on single layers of permissive cells.

In order to create recombinant adenovirus with SAG1 gene (AdSAG1) plasmid pJM17 and the transference vector pCMVlink1-SAG1 were transfected. For the construction of the recombinant adenovirus with SAG2 gene (AdSAG2) the pJM17 and the transference vector pCMVlink1-SAG2 were co-transfected. For the construction of the adenovirus containing the SAG3 gene (AdSAG3) the plasmid pJM17 and the transference vector pMV60-HASS-SAG3 were co-transfected.

For occurrence of the transfection (co-transfection) occurrence, a first procedure was necessary for the preparation of permissive cells. During this first procedure of preparation of permissive cells, as for instance, HEK293A cells were sowed preferably in 6 wells plates, at a density of approximately 600,000 cells/well. At each well approximately 3 ml/well of HEK293A cell was added, which cells were cultivated approximately 24 hours at a preferred temperature of 37° C. and in an atmosphere with approximately 5% of CO₂, in an adequate culture medium, as for instance the DMEM mean, which was supplemented with of approximately 5% of bovine fetal serum, sodium bicarbonate at a preferred concentration of 5 mM, HEPES at a preferred concentration of 25 mM and approximately 40 mg/l of gentamicin. This medium and culture is called a complete DMEM medium.

In order to generate each one of the adenovirus, at least one co-transfection response was performed. Each co-transfection response was performed in an individual well of a 6 wells plate, containing a single layer of approximately 600,000 cells.

After approximately 24 hours of cultivation, the single layers of permissive cells, present in the wells of the plate were washed, at least once, with approximately 1 ml/well of an adequate culture medium, as for instance the complete DMEM medium, in such a way that all dead cells present in the wells were removed. Until the moment of addition of transfection reagents and of the plasmids, the 6 wells plates containing the washed single layers were maintained in cultivation in approximately 3 ml of complete/well DMEM medium, at a preferred temperature of 37° C. and at an atmosphere of 5% of CO₂.

In order to generate the AdSAG1, the following co-transfection response was assembled: approximately 5 μg of the pJM17 plasmid and approximately 5 μg of the pCMVlink1-SAG1 transference vector were diluted together in preferably of-ionized water and sterile qsp 450 μl.

Approximately 50 μl of a sterile solution of CaCl₂ at a preferred concentration of 2.5M were added to this mixture. In a preferably conic tube of polypropylene approximately 500 μl of an adequate tampon solution was added. In this substantiation we used a tampon solution HeBS 2×, in order to maintain the pH value within 7.05-7.12 range. The referred tampon solution is composed of sodium chloride at the preferred concentration of 280 mM, HEPES at the preferred concentration of 50 mM and dibasic sodium phosphate preferably at 1 mM.

Approximately 500 μl of mixture containing the pJM17 vector, the pCMVlink1-SAG1 vector and the CaCl₂ previously prepared were added, drop-by-drop in the 500 μl of the HeBS tampon divided in the polypropylene tube. The HeBS tampon was maintained under constant agitation, through air injection, during all the process of dripping of the plasmid and CaCl₂ mixture. Approximately 1000 μl of the plasmids, CaCl₂ and HeBS tampon mixture, denominated transfection response, was left to rest during approximately 30 minutes in a preferred temperature of 25° C., in order to allow the formation of the precipitate. The referred precipitate consists of calcium phosphate crystals associated to a plasmid DNA.

After the incubation period, the referred transfection response was agitated in adequate equipment, as for instance a “vortex”, and approximately 3 ml of the complete DMEM culture medium were added to the referred transfection response. The approximately final 4 ml of transfection response added with complete DMEM was immediately transferred to a well of one cultivation plate containing approximately 600,000 HEK293 permissive cells. The complete DMEM medium, in which the cells were being maintained in cultivation was duly removed of the well before the addition of a transfection response in the new medium.

For the generation of the AdSAG2 the following response of co-transfection was assembled: approximately 5 μg of the pJM17 plasmid and approximately 5 μg of the pCMVlink1-SAG2 transference vector were diluted together in preferably deionized water and sterile qsp 450 μl.

Approximately 50 μl of a preferably sterile solution of CaCl₂ at a preferred concentration of 2.5M was added to this mixture. In a preferably conic polypropylene tube approximately 500 μl of adequate tampon HeBS 2× solution was added.

Approximately 500 μl of mixture containing pJM17 vector, pCMVlink1-SAG2 vector and CaCl₂ previously prepared was added, by dripping into the 500 μl of the HeBS tampon divided in the polypropylene tube. The HeBS tampon was maintained under constant agitation, through air injection, during the whole dripping process of the mixture of plasmid and CaCl₂.

The approximately 1000 μl of the resulting transfection response were left to rest for approximately 30 minutes at a preferred temperature of 25° C. so that there was he formation of a precipitate of calcium phosphate crystals associated to a plasmid DNA.

After the incubation period of the mixture, it was agitated in a “vortex” and approximately 3 ml of the complete DMEM medium of culture was added. The approximately 4 ml of the transfection response added by complete DMEM was immediately transferred to a well of a cultivation plate containing approximately 600,000 cells. The complete DMEM medium, where the cells were being maintained in cultivation was duly removed from the well before the addition of the transfection response in the new medium.

For the generation of the AdSAG3 the following co-transfection response was assembled: approximately 5 μg of the pJM17 plasmid and approximately 5 μg of the pMV60-HASS-SAG3 transference vector were diluted together in preferably deionized water and sterile qsp 450 μl. To this mixture, approximately 50 μl of a sterile solution of CaCl₂ at a preferred concentration of 2.5M was added.

In a preferably conic polypropylene tube, approximately 500 μl of tampon HeBS 2× solution was added. Approximately 500 μl of a mixture containing the previously prepared pJM17, the pMV60-HASS-SAG3 and CaCl₂ was added by dripping at the 500 μl of the HeBS 2× tampon divided in the polypropylene tube.

The HeBS tampon 2× was maintained under constant agitation, through air injection, during the whole dripping process of the mixture of plasmids and CaCl₂. Approximately 1000□1 of the resulting transfection response was left to rest for approximately 30 minutes in a preferred temperature of 25° C. so that there was the formation of a calcium phosphate precipitate associated to a plasmid DNA.

After the incubation, the referred transfection response was agitated in a “vortex” and approximately 3 ml of complete DMEM was added. The approximately final 4 ml transfection response added by complete DMEM was immediately transferred to a well on a cultivation plate of approximately 600,000 cells. The complete DMEM medium, in which the cells were being maintained, was duly removed of the well before the addition of the transfection response in the new DMEM medium.

The cultivation plate containing the single layers of permissive cells added to the transfection responses were cultivated with the said reactions during the preferred period of 16-18 hours, to allow the caption of the plasmids.

After the period of preferred cultivation of 16-18 hours, the wells of the plate were preferably washed twice with an adequate solution, as for instance, a solution of PBS to allow the removal of the excess of the precipitate.

Permissive cells were treated with a solution of trypsin, preferably divided in to the of 1:3 ratio and cultivated again in an adequate culture medium, as for instance, in a 4 ml/well of a complete DMEM culture medium, at an approximate temperature of 37° C. and preferred atmosphere with 5% of CO₂. Permissive cells were preferably cultivated until the moment of the development of an homologue intracellular recombination of the plasmids in approximately 4 ml/well of complete DMEM, at an approximate temperature of 37° C. and a preferred atmosphere with 5% of CO₂. During this period, the 4 ml of the complete DMEM of the wells were substituted for approximately 4 ml of new complete DMEM, and this exchange was performed in approximately each 72 hours.

The formation and a replication of recombinant adenovirus in the cultivation of permissive cells were evidenced by the appearance of a cytopathic effect and formation of the plates of lysis in the cell single layer.

After the homologue recombination, generation and replication of recombinant adenovirus, occurs the formation of the plates of lysis in the single layers of permissive cells and consequent release of viral recombinant particles to the cultivation medium. At this stage, such cultivations of infected permissive cells become called primary cultivations of recombinant adenovirus. Approximately 4ml of overflowing material of these primary cultivations of adenovirus were collected and used for the first amplification of recombinant adenovirus.

During the period of preparation of permissive cells for the first amplification of the adenovirus, the referred overflowing material of the primary cultivations was preferably stored in 2 ml portions, approximately at −70° C., in polypropylene tubes, without the addition of preservers.

For the first amplification of each recombinant adenovirus, a bottle of 25 cm² of cultivation was sowed with permissive HEK293 cells in 7 ml of DMEM complete medium and cultivated preferably at 37° C. and with 5% of CO₂.

When a single layer at the bottle reached around 90-95% of confluence, the process of infection of the cells of the single layer with the viral particles contained in the overflowing material of primary cultivations was performed.

For the referred infection, the old 7 ml DMEM complete medium was removed from the bottle and substituted for approximately 2 ml of overflowing material obtained in the primary cultivation. The bottle was then incubated for approximately 60 minutes at a temperature of approximately 37° C., without ventilation.

After this period, the volume of the bottle was completed to approximately 7 ml, adding approximately 5 ml of new complete DMEM medium. The 25 cm² infected bottles, hereby called secondary cultivations, were cultivated during approximately 48 hours at a temperature of approximately 37° C., without ventilation.

After this period, all cells were infected and were released from the bottom of the bottles. The content of the secondary cultivation bottles was stored in portions of approximately 7 ml, polypropylene tubes and without the addition of preservers, preferably at −70° C.

For the second amplification of each recombinant adenovirus, 1 bottle of 175 cm² of cultivation was sowed with HEK293 permissive cells in 35 ml of a complete DMEM medium and cultivated preferably at 37° C. and with 5% CO₂.

When a single layer in the bottle reached approximately 90-95% of confluence, the process of infection of cells of single layer was performed with the overflowing material of the secondary cultivations.

For this infection, the old complete 35 ml DMEM medium was removed from the bottle and substituted for approximately 7 ml of overflowing material obtained from secondary cultivations plus 7 ml of the new complete DMEM medium. The bottle was then incubated for approximately 60 minutes at a temperature of approximately 37° C., without ventilation.

After this period, the volume of the bottle was completed to approximately 40 ml, adding 25 ml of the new complete DMEM medium. The infected bottle was cultivated during approximately 48 hours at a temperature of approximately 37° C., without ventilation.

After this period, all cells were infected and released from the bottom of the bottles. The 175 cm² overflowing material obtained in the bottle was preferably stored in 40 ml portions, at approximately −70° C., in polypropylene tubes, without the addition of preservers.

For the large scale amplification of each adenovirus and the production of purified viral stocks, HEK293A permissive cells were preferably sowed in 12 bottles of cultivation of 175 cm², which contained an adequate culture medium, as for instance, the DMEM complete medium. These permissive cells were cultivated preferably in 35 ml of complete DMEM, at 37° C. and in an atmosphere with 5% CO₂ until reaching approximately 90-95% of confluence.

After the permissive cells having attained approximately 90-95% of confluence, the infection of these cells was performed.

For the procedure of infection, 40 ml of the overflowing material obtained in a previously infected bottle of cultivation of 175 cm² was diluted in 140 ml of new complete DMEM medium. Approximately 35 ml of the old complete DMEM medium was removed of the bottles of cultivation of 175 cm² and substituted for 15 ml of diluted overflowing material.

The 12 bottles were incubated for 60 minutes at approximately 37° C., without ventilation. After this period, the volume of the bottle was completed to approximately 40 ml, adding 25 ml of new complete DMEM medium. The infected bottles were cultivated during approximately 48 hours at a temperature of approximately 37° C., without ventilation.

After the period of incubation for the appearance of the cytopathic effect and the loosening of permissive cells, the content of the bottles of incubation was collected and centrifuged at approximately 300 g during approximately 10 minutes, at a preferred temperature of 4° C.

After the centrifugation stage, permissive cells were resuspended in an adequate tampon solution, as for instance, a Tris 0.1M pH=8.0 tampon solution and preferably lysated with a solution of sodium deoxicolate at 0.5%, during approximately 30 minutes, in an ice bath.

Afterwards, the extract of permissive cells obtained during the last stage was manually homogenized with a glass homogenizing apparatus and approximately 10 mL of the this extract was added to an approximate volume of 5.8 mL of a solution saturated preferably with cesium chloride.

The new mixture obtained was centrifuged at approximately 100,000 g during approximately 16 hours to allow the banding of viral particles. After the centrifugation of the new mixture, the adenovirus suspension was collected and desalinized through an adequate technique. In this substantiation we used the dialysis technique.

The dialysis performed was made against an adequate tampon solution adequate, as for instance a tampon solution Tris 0.01M, for approximately 4 hours, at a preferred temperature of 4° C. The purified virus obtained after the dialysis were preferably frozen with 10% of glycerol at a preferred temperature of −70° C.

During the virus titulation stage in permissive HEK293A cells, these cells were preferably sowed in 24 wells plates, at a density of approximately 300,000 cells/well. The plate contained, within its wells, an adequate culture medium, as for instance, the complete DMEM culture medium. Cells were cultivated during approximately 24 hours, at a preferred temperature of 37° C. and in an atmosphere with approximately of 5% CO₂.

The stock of virus obtained during the replication and purification stages were preferably diluted in series, preferably cultivation plates of 96 wells, with approximately 225 μl of complete DMEM medium per well, with a preferred dilution factor equal to 10. Each one of the dilutions was performed four times, and approximately 200 μl of each one of the replicas of dilutions 10⁻⁷ to 10⁻¹² were used to infect a well with 300,000 permissive cells.

The infection was made through incubation of the plates of cultivation of the 24 wells, with approximately 200 μl per well of the mentioned infections of adenovirus, during approximately 60 minutes at a temperature around 37° C., in a preferred atmosphere of 5% of CO₂. After this incubation, the volume of the medium of each well was increased to 1.7 ml through addition of 1.5 ml of a new complete DMEM.

The cultivations were maintained at an approximate temperature of 37° C. and in approximately 5% of CO₂, during approximately 7 days until the time of appearance of the plates of lysis in the single layer of cells. The title was calculated as of the highest dilution of virus, which lead to the formation of the plate of lysis and the referred title was expressed in number of units forming the plate of lisys per milliliter of viral stock (ufp/ml).

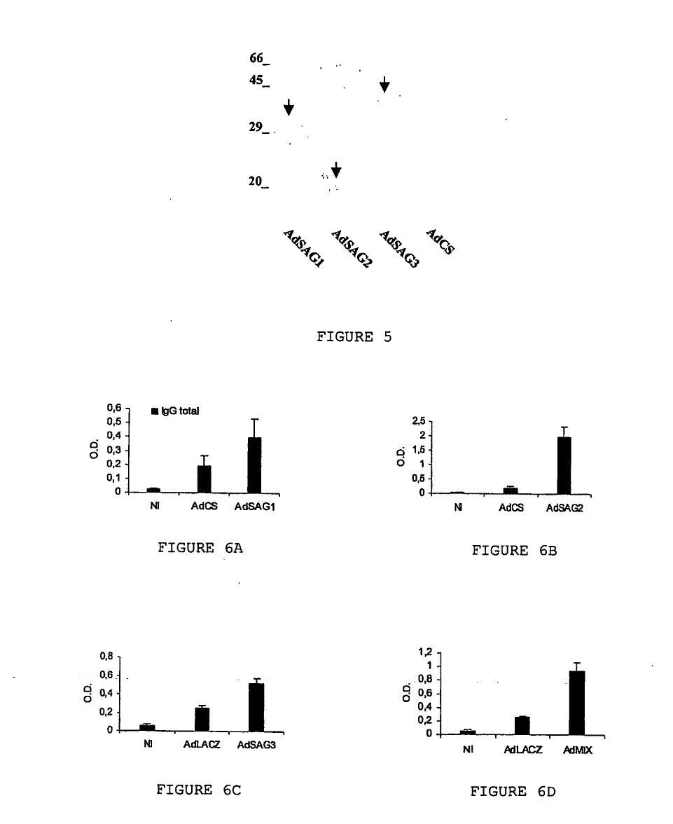

The expression of SAGs different proteins with the recombinant adenovirus was evaluated “in vitro” through the “Western-blot” technique performed for the extract of permissive cells, HEK293A infected against the serum of an immunized rabbit with a total antigen of T. gondii. As shown in FIG. 5, we observe that the virus are able to induce the expression of single proteins, as which promptly react with the serum of the immunized animal.

Now, this invention shall be described in detail through the example. It is necessary to stress that the invention is not limited to this example, but it also includes variations and modifications within the limits in which it functions.

### EXAMPLE

### Evaluation of Immunogenic Capacity of Adenovirus with Genes of Proteins SAG1, SAG2 and SAG3 in BALB/c Rats

Recombinant adenovirus with the genes of proteins SAG1 (AdSAG1), SAG2 (AdSAG2) and SAG3 (AdSAG3) were submitted to an evaluation of their immunogenic capacity in BALB/c rats in an immunization plan, where the initiation and the homologue (prime/boost) reinforce are involved. In this immunization plan, preferential groups of 10 females with approximately 6-8 weeks of age received preferably two subcutaneous applications of approximately of 10⁹ pfu of adenovirus preferably with six weeks of interval between applications.

In each experiment of immunization, 6 vaccination groups were organized: a first group vaccinated with AdSAG1, a second group vaccinated with AdSAG2, a third vaccinated with AdSAG3, a fourth group that received a mixture of AdSAG1, AdSAG2 and AdSAG3 in equivalent quantities (AdMIX), a fifth group that received a control adenovirus, and in this substantiation, the adenovirus applied to the control group were AdCs adenovirus, which codify for protein CS of Plasmodium yoelii or the AdlacZ adenovirus, which codifies for β-galactosidase, and a group that did not receive any kind of inoculation. Experiments of immunization with this composition of groups were repeated twice.

The production of IgG anti-SAG antibodies was evaluated in samples of serum obtained in bleeds made approximately 10 days after the completion of the immunization plan.

ELISA tests were performed against purified antigens of the membrane of the tachyzoites form and “western-blot” against the total extracts of the tachyzoites form of RH strain of T. gondii.

FIGS. 6A, 6B, 6C and 6D show the comparison of the results of ELISA tests. We observe a significant increase in the reactivity (O.D.) of the serum of immunized animals in face of the antigens of the protozoa T. gondii, related to the presence of the IgG antibody. We inform that for FIGS. 6A, 6B, 6C and 6D, the nomenclature NI means that the animals were not immunized; the nomenclature AdlACZ represents animals immunized with adenovirus that express β-galactosidade; the nomenclature AdCS represents animals immunized with adenovirus that express the CS protein of the Plasmodium yoelii; the nomenclature ADMIX represents animals immunized with a mixture of AdSAG1, AdSAG2, AdSAG3. Additionally, we detected a higher level of production of antibodies of the IgG2a subtype in relation to the IgG1 antibody. This production level is a sign of the activation of a Th1 type immune response against antigens codified by the adenovirus. The specificity of these antibodies is confirmed through a response of “Western-blot”, in which the serum of immunized animals reacts with native forms of antigens in total extract of tachyzoites of T. gondii, as shown in FIG. 6.

This immunization plan allows a preliminary evaluation of the protecting capacity of the recombinant adenovirus. In this evaluation, immunized animals were challenged with the strain P-Br of T. gondii, which is known to cause chronic infection in rats, follows by the formation of tissue cysts in these animals. The P-Br strain is classified by its characteristics as a cystgenic strain.

In this procedure for evaluation, preferable at the 14^{th} day after the immunization, each one of the animals received an oral dose of approximately 20 cysts of strain P-Br and approximately 45 days after the ministration of this dose, the animals were sacrificed.

After the sacrifice of the animals, the brain of the animals was collected and there was a counting of the number of cysts present in the brain. In order to perform the counting of cysts, the brain of each rat was individually macerated in 1 ml of PBS tampon. The maceration was manually done using glass conic tubes of 5 ml and glass sticks.

For counting, two portions of 10 μl of each digest of the brain were glued on glass blades appropriate for microscopy and covered with small glass blades with preferred area of 22 cm×22 cm. The counting of cysts was made in an optical microscope, under an enlargement of 400×, within the whole area covered by the small blades.

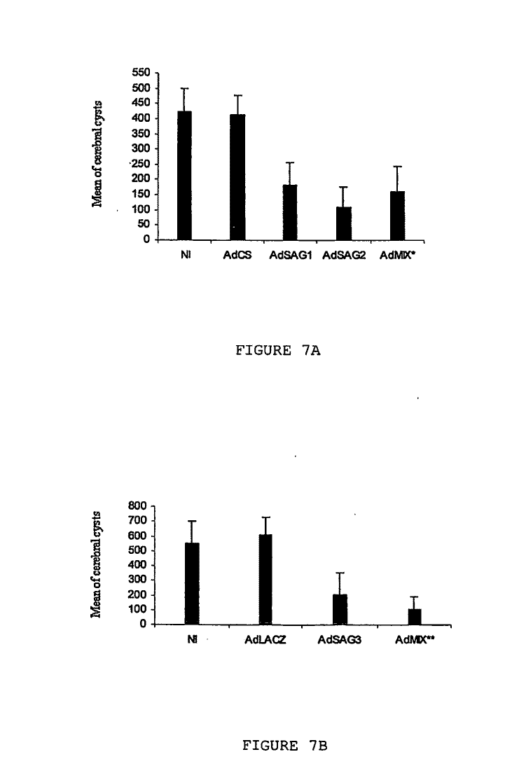

As a result of the referred counting, it was observed that the immunization plan for the protozoa T. gondii used provided a significant reduction in the load of cerebral cysts. In independent experiments, the level of reduction in the load of cysts was approximately 50 to 60% for AdSAG1, approximately of 60 to 70% for AdSAG2, approximately of 70% for AdSAG3 (a single experiment until now) and approximately of 80% for the combination of the 3 adenovirus (a single experiment until now). FIG. 7A and FIG. 7B show the results of the count of the number of cysts present in the brain, obtained in the experiment with AdSAG1, in the experiment with AdSAG2, in the experiment with AdSAG3 and in the experiment with the combination of the three vectors.

The hereby-described invention, as well as the features approached should be considered as one of the possible substantiations. It must, however, remain clear that the invention is not limited to this substantiations and that, with ability in techniques, we shall perceive that any particular characteristic introduced to it must be only understood as something that was described to facilitate the understanding and that they cannot be made without departing from the creative concept described. The limiting characteristics of the object of this invention are related to the claims that are part of his report.

1. Process for production recombinant adenovirus with genes that codify for T.gondii SAG1, SAG2 e SAG3 proteins, RH strain, characterized by comprising the stages:
cloning of adenovirus genes that codify SAG1, SAG2 and SAG3 proteins without motive for the addition of glycophosphatidylinositol (GPI)anchor in the transference vector;
co-transfection of transference vectors and of the genome of the adenovirus;
intracellular homologue recombination between the transference vectors and the genome of the adenovirus;
amplification of the recombinant viral genome viral
purification of viral particles into an adequate gradient;
characterization of the expression of the proteins of interest.

2. Process according to claim 1, characterized by the presence of the recombinant gene be confirmed by the digestion profile and posterior sequencing of the purified plasmid, the expression of SAG1 e SAG2 protein being determined through technique of transfection of eucariote cells, followed by detection of proteins in the lysate of the transfected cells, through “Western-blot” technique.

3. Process according to claim 1, characterized by the process of cloning of SAG3 gene be differentiated due to the substitution of the original signal peptide of the gene for a viral sign peptide.

4. Process according to claim 3, characterized by the plasmid pcDNA3.1, receptor of the SAG3 gene be prepared through the introduction of a DNA fragment that codifies the signal peptide of haemagglutinin of the influenza virus (HASS) within the expression cassette of the referred plasmid.

5. Process according to claim 3, characterized by the presence of the SAG protein gene be evaluated through reactions of digestion and posterior sequencing of plasmids, the expression of the SAG3 protein be evaluated through transfection of permissive cells, detection of the protein in the cell lysate through “Western-blot” technique.

6. Process according to claim 1, characterized by the generation of the recombinant adenovirus with SAG1 (AdSAG1) gene occur through co-transfection of pJM17 plasmid and the transference vector pCMVlink1-SAG1, the SAG2 gene (AdSAG2) occur through the co-transfection of the pJM17 plasmid and the transference vector pCMVlink1-SAG2 and SAG3 (AdSAG3) gene occur through transfection of pJM17 plasmid and transference vector pMV60-HASS-SAG3.

7. Composition of the vaccine using recombinant adenovirus obtained as claimed in any of the previous claims, characterized by the viral structure being modified as of the insertion of exogenous DNA fragments, substituting the original E1 region of the human type 5 adenovirus genome.

8. Composition according to claim 7, characterized by the administration of recombinant adenovirus to be performed at least twice through subcutaneous application.

9. Composition according to claim 7 e 8, characterized by the occurrence of a significant increase of the (O.D.) reactivity of serum of immunized animals face the antigens of the T. gondii protozoa, related to the presence of IgG antibodies.

10. Composition adenovirus according to claim 7 e 8, characterized by fact that the immunization plan for the T. gondii protozoa used provides a reduction on the load of cerebral cysts of 50 to 60% for AdSAG1, approximately 60 to 70% for AdSAG2, approximately 70% for AdSAG3 and approximately 80% for the combination of the 3 adenovirus.
